(19) United States
(12) Patent Application Publication
(10) Pub. No.: US 2005/0059077 A1
(43) Pub. Date: Mar. 17, 2005

(54) Interfering with telomere maintenance in treatment of diseases

(75) Inventors: Stephen Friend, Seattle, WA (US); Patrick Paddison, Oyster Bay, NY (US); Melisa Dashiel, Everett, WA (US); Antonio Bedalov, Seattle, WA (US)

Correspondence Address: MCDERMOTT WILL & EMERY LLP, 600 13TH STREET, N.W., WASHINGTON, DC, 20005-3096, US

(73) Assignee: Fred Hutchinson Cancer Research Center, Seattle, WA (US)

(21) Appl. No.: 10/973,461

(22) Filed: Oct. 27, 2004

Related U.S. Application Data
(63) Continuation of application No. 09/678,952, filed on Oct. 3, 2000, now abandoned.
(60) Provisional application No. 60/156,938, filed on Sep. 30, 1999.

(51) Int. Cl. C12Q 1/68

(52) U.S. Cl. 435/6

(57) ABSTRACT

The invention relates to novel nucleotide sequences and the polypeptide sequences derived therefrom, which are involved in yeast and mammalian telomere regulation. Methods for screening drugs and compositions are also provided. The invention furthermore relates to methods involving a diagnostic and/or therapeutic use of these gene(s)/protein(s), principally for treating cancer and aging.

### 1. FIELD OF THE INVENTION

This invention relates to compositions and methods of identifying eukaryotic genes, which are important in regulating cell cycle. More specifically it relates to yeast genes and their mammalian counterpart genes that are essential in cell growth, aging, and death. Methods and compositions are provided for identifying and modulating cell cycle related genes and their respective functions responsible for longevity or malignancy.

### 2. BACKGROUND OF THE INVENTION

Genetic manipulation of the yeast genome provides a convenient model for identifying essential genes required for eukaryotic cell replication, growth, and ultimately aging or senescence. The events related to cell longevity and cancerous cell immortalization are closely related. In order to prevent aging or cancer growth, one must first find causes and means of regulating them on a molecular level.

About 40 years ago Leonard Hayflick and Paul Moorhead first noticed that fibroblasts can divide only a limited number (50-60) of times—a phenomenon now known as “Hayflick's Limit”. In contrast to normal cells, malignant cells can divide an infinite number of times. Most recent research efforts in the gerontology/oncology area has been focused on telomeres. In broad terms “telomeres” are the tips of the chromosomes, composed of repeated nucleic acid sequences but containing no genes. These tips are shortened at each division, which causes genetic imbalances. It is known that cancer cells, germ cells, and all of studied eukaryotic microorganisms have the ability to correct this shortening with an enzyme called telomerase. The control of senescence by telomerase or its known genes (TLC1 and EST) is still being investigated, with results being preliminary and often contradictory. Nevertheless, it has been shown that telomere loss may play a role in senescence, a scenario for which there is evidence in S. cerevisiae (see e.g., U.S. Pat. Nos. 5,695,932; 5,489,508; 5,840,495). The loss of telomeres might not be the only cause of what is called aging; the loss may also portend the onset of other pathological conditions, which may manifest themselves later in life—conditions such as Creutzfeldt-Jacob disease, Alzheimer's disease, wearing of joints, presbyopia, programmed cell death, cancer, etc., may contribute to aging. Whether these conditions are considered diseases or aging proper is a matter of definition. In general, all lethal genes are opponents to the host's prolonged survival and all of them are worthy of attention. It is likely that there are many genes in a host genome that are responsible for the aging process or tumorigenesis although they are not necessarily obvious, at this time point, as being involved in aging or cancer.

Many important scientific discoveries that eventually benefited the human host were first made in a model system. Since the complete DNA sequence of the yeast Saccharomyces cerevisiae strain S288C was determined in April 1996, baker's yeast has become a very convenient model for elucidating problems of aging or cancer on a genetic level. Databases containing yeast genome sequences are available publicly and can be found for example on public websites such as for example: http://bioinformatics.weizmann.ac.il; http://genome-www.stanford.edu/Saccharomyces/; http://vectordb.atcg.com/vectordb/; http://www.mpimg-berlin-dahlem.mpg.de/˜andy/GN/S.cerevisiae/; or http://www.mips.biochem.mpg.de/proj/yeast/. Other databases exist as well and these databases and links therein to other websites are equally suitable for the purposes of this invention. The examples include but are not limited to Yeast GenBank (a collection of all GenBank sequences that were derived from Saccharomyces cerevisiae); Yeast Swiss-Prot (the collection of Swiss-Prot protein sequences that are derived from Saccharomyces cerevisiae); and YPD (the Yeast Protein Database maintained by Proteome, Inc., the contents of which are incorporated herein by way of reference. Methods of manipulating yeast are well established and are well known to those skilled in the art and can be found in publicly available web sites such as for example www.goshen.edu/bio/yeast, www.fhcrc.org/˜gottschling, and www.sacs. ucsf.edu/home/HerskowitzLab, among many others.

While the sequence of all yeast genes is known and mapped on their respective chromosomes it is still difficult to predict the biological function of many of these genes especially those that have no counterparts in other species or fail to reveal a sufficient sequence homology with known genes. In other words determining the sequence of a gene is easier then ascribing a meaningful function to a gene. Furthermore, even with genes having known functions it is not obvious how the function or regulation of such a gene may be modified when combined with another gene's regulatory mechanism or product. Thus, the art is still highly unpredictable when it comes to the problem of identifying a gene's known or unknown function in combination with another gene.

This problem is usually solved on a case by case basis, whereby investigators apply known gene manipulation techniques and screening methods to each specific gene of interest or particular gene combination. Yeast is often selected as a model due to the ease of manipulation and possibility of screening a large number of candidates in a relatively short period of time. Yeast has the highest rate of recombination and gene conversion among organisms tested, which is several orders of magnitude higher than in mammals. In the post-genomic era, serial gene-knockout studies in yeast (which you can tell by tetrad analysis, e g., http://bioinformatics.weizmann.ac.il/pub/software/mac/mactetrad 69.readme) revealed that about 1 in 6 gene products are essential to the life of that cell (their deletion is lethal) under tested conditions. While a reasonable fraction of tested gene products are enzymes, other genes have unknown function or their function does not fit into apriori postulation.

A commonly used approach is screening by gene knockout and complementation. The term “complementation” is used herein as a genetic term intended to mean that the subject genetic element is homologous to a mutant genetic element such that when introduced into a cell it rescues the cell from the effects of the mutation (see e.g., U.S. Pat. No. 5,527,896). For further details and examples see U.S. Pat. No. 5,866,338 to Hartwell, et al., incorporated herein by way of reference. Findings in yeast are readily translatable to human gene(s) regulation since many of yeast genes are homologous to human counterparts. Means of cross-referencing the yeast and human genes are now achievable and can be for example performed using “XREF2” program as found on http://www.ncbi.nlm.nih.gov/XREFdb/, which is incorporated herein by way of reference.

Some of these strategies resulted for example in the identification of DNA replication accessory factors from the yeast Saccharomyces cerevisiae, which later were found to be reproducible in the mammalian host.

Several US patents exist which provide an insight into state of the current art on telomerase and its function. For example, incorporated by reference, U.S. Pat. Nos. 5,916,752 and 5,698,686 disclose telomerase compositions and screening methods; U.S. Pat. Nos. 5,958,680 and 5,917,025 disclose telomerase genetic sequences; and various U.S. Pat. Nos. 5,916,752; 5,891,639; 5,888,747; 5,876,979; 5,863,936; 5,863,726; 5,859,183; 5,858,777; 5,856,096; 5,846,723; 5,840,495; 5,840,490; 5,837,857; 5,837,453; 5,830,644; 5,804,380; 5,776,679; 5,770,613; 5,767,278; 5,760,062; 5,747,317; and 5,733,730 disclose other related and unrelated aspects of telomerase and telomeres. None of them however provide an insight into instant discovery.

Thus, until the present invention no reports existed in the prior art suggesting or teaching the existence of instant genes/proteins involved in telomere/telomerase regulation in yeast or in eukaryotic cells in general.

### 3. SUMMARY OF THE INVENTION

It is an object of this invention to provide yeast mutants as models of tumor cells or other deregulated cells corresponding to various diseases.

According to the present invention it is now found that genetic screening, e.g., by synthetic lethality, in yeast reveals novel genes involved in telomerase pathway. More specifically, mutations in three recessive yeast genes tol1, tol2, tol3 (telomerase overexpression lethal) have been identified that render unviable host cells with an overexpressed telomerase gene TLC1 (RNA component of telomerase such as found in S. cerevisiae). By “genes” is meant sites, domains, actual genes, sequences, sections, fragments, or open reading frames, which have been identified by the present method in the genome of a cell, mutant alleles of which prove to be lethal to the cell, especially the cell exhibiting aberrant telomerase activity. By aberrant telomerase activity is meant elevated telomerase activity in a cell relative to a normal cell. Such elevation of telomerase activity may be brought about by overexpression of a telomerase gene by, for example, changes in the promoter region of the gene, duplication(s) of the gene, translocation or loss of chromosomal material resulting in the aberrant fusion of the coding region of a telomerase gene with a more active promoter. Elevated telomerase activity may also be brought about by mutations in the coding region resulting in a more active form of protein. Analogs and homologs of TLC 1 are also contemplated as preferred embodiments of the invention including but not limited to embZ35904; gbU14595SCU14595; embZ35905SCYBR036C; dbjD28120YSCCLS2; gbL24113YSCCSG2P; embX76992SCPDX3; in Homo sapiens clone DJ0261D10 gbAC005476.3AC005476; gbU53340CELF02E8 in Caenorhabditis elegans. Similarly analogs and homologs of EST gene series or products thereof (“ever shorter telomeres” or catalytic component of telomerase) are preferred including but not limited to human kininogen HMW heavy chain, prepro alpha-2-thiol proteinase, calmodulin-stimulated protein, kininogen, immunoglobulin kappa chain, nitric-oxide synthase, immunoglobulin heavy chain variable, T-cell receptor delta-chain V, Ig gamma-chain, Ig H-chain V-D-JH4-region, perlecan, insulin-like growth factor II, interferon-alpha, rat coding sequence of p15 and p12, interferon-alpha I precursor, AAD10, among many others.

Furthermore a mammalian and/or human homolog of tol gene(s) are contemplated which play a similar role in telomere regulation and consequently in aging and cancer process. This is validated in human cell lines (fibroblasts and epithelial cells with or without hTERT—closest homolog of yeast EST2).

As a result of this insight into the mechanism of regulation of instant genes, means of screening for drugs useful for treating and preventing cancer are also provided. Without limiting to aforementioned category of diseases this invention also provides means for treating various mycotic or yeast pathogens. By mass screening and genetic analysis compounds and/or drug targets are identified that could specifically kill eukaryotic organisms that over-express telomerase. Accordingly, therapeutic agents are contemplated, which are developed from the identification of essential genes of eukaryotic organisms. Such identified genes or products thereof serve as novel targets for therapeutics based on a mechanism which is distinct from to the known mechanism(s) of existing drugs. Such a compound inhibits or enhances the function of a gene or gene product identified by methods disclosed herein, for example, by producing a phenotype or morphology similar to that found in the original mutant strain.

In a particular embodiment of the invention, the primary gene defect is preferably one found in or associated with a tumor cell or cell affected with cell cycle deregulation. Alternatively, the primary gene defect in the cell provided by the instant invention is analogous or homologous to a defect found in or associated with a mammalian or human tumor cell or chromosomally aberrant cell. By “homologous” is meant a direct relationship among a “family” of genes in which certain sequences or domains are strongly conserved (at least 40% sequence identity) among the members of the family. On the other hand, “analogous” genes may serve similar or “analogous” functions, but they are not directly related (i.e., sequences are not conserved among analogous genes).

As a result of this insight into the mechanism of regulation of instant genes means of diagnosing, treating and preventing senescence are provided. The present invention relates to methods and reagents for extending the life-span, e.g., the number of mitotic divisions, of a cell.

A further object of this invention includes means of gene therapy by gene product in a cell population that contains at least one primary gene defect, wherein the exposure of the cell population to the therapeutic gene arrests or kills subject cell selectively in the cell population. Such gene products are encoded or regulated by a human gene analogous or homologous to yeast tol gene.

A synthetic lethal screening method is disclosed based on lethality of yeast mutants having known overexpressed primary gene of interest and secondary mutated genes, which in combination determine cell death. The subject screening assay uses multiple mutants that are capable of complementing or suppressing the function of genes of interest. Experimental examples enabling this invention are provided which identify novel genes involved in cell cycle control in most general terms.

A method of identifying a compound useful in the treatment of proliferative diseases, such as cancer, characterized by the overexpression of telomerase activity which comprises the steps of 1) contacting a tol gene product or an analog or homolog thereof with a test compound under conditions and for a time sufficient to permit the test compound to effect the tol gene product; 2) comparing the activity of the tol gene product in the presence of the test compound with the activity of the gene product in the absence of the tol gene product. Within this exemplary assay, compounds that can decrease the activity tol gene product or an analog or homolog thereof are identified as useful for the treatment of cancer or other proliferative diseases characterized by overexpression of telomerase activity. Within another embodiment of the invention, whole cell assays are conducted wherein a cell with a wildtype tol gene or analog or homolog thereof is contacted with a test compound and the expression and/or activity of the tol gene product or analog or homolog thereof is assessed and compared with the expression and/or activity of the tol gene product in cells not exposed to the test compound. Within this aspect, those compounds that result in decreased expression or activity of the gene product are useful for the treatment of cancer or other proliferative diseases characterized by overexpression of telomerase activity.

Transgenic nonhuman organisms, such as transgenic mice, with tol mutants are also provided. Methods of using these organisms, including methods of detecting compounds that play role in controlling telomerase overexpressing animals are also disclosed.

A further object of this invention provides for method of inhibiting the growth or replication of a tumor cell or causing the demise of the cell exhibiting aberrant telomerase activity, comprising administering a drug or drug candidate that interacts with, binds to, or inhibits the expression or activity of a gene product associated with a secondary target site in the genome of said cell, which site can accommodate at least one mutation that can prove lethal to the cell.

### 4. DETAILED DESCRIPTION OF THE INVENTION

This invention provides genes and their respective products that are essential in the presence of high telomerase activity and dispensable in its absence. The purpose of this study is to isolate the mutants that are inviable in the presence of high level of telomerase (TOL telomerase overexpression lethal) but are able to grow in its absence. This insight provides a powerful means for controlling undesired cell growth (tumor) and also provides means to enhance the lifespan of a normal cell.

The scheme of the screening method is outlined below. General technical procedures in manipulating yeast are found in C Guthrie and G R Fink. Methods in Enzymology. (Volume 194: Guide to Yeast Genetics and Molecular Biology, 1991, Academic Press) the content of which is incorporated herein in its entirety. A mutant yeast overexpressing galactose inducible EST1, EST2 and TLC1 with markedly (3-4 fold) elongated telomeres is used in the screen. This strain expresses the EST1 and EST2 genes from the chromosomes while the GAL1-TLC1 is provided on a URA3 containing plasmid.

Twenty four hours prior to ethyl methanesulfonate (EMS) mutagenesis, the strain is shifted to glucose (TLC1 off) to eliminate telomerase activity. After mutagenesis, cells are plated on a selective medium containing glucose. Colonies are replica plated on three different plates: selective plates containing glucose (TLC1 off), selective plates with galactose and raffinose (TLC1 on) and 5-FOA [See, Boeke, J. D., et al. 5-Fluoroorotic acid (5-FOA) as a selective agent in yeast molecular genetics. Methods Enzymol. (1987) 154:164-1751 plates with galactose and raffinose. Mutants which can not tolerate telomerase activity are detected by the lack of growth on galactose. If the failure to grow on a galactose is, in fact, caused by telomerase, cells that have lost the plasmid are able to grow on galactose medium containing 5-FOA. Colonies that fit the described growth pattern are then mated with the wild type strain with inducible EST1, EST2 and TLC1 genes. If the mutation is recessive, the diploid tol/TOL strain is viable and able to tolerate high telomerase activity. The strain is then sporulated and the spores are analyzed. If the mutation is dominant (TOL-dom) it is not possible to induce telomerase in the TOL-dom/TOL diploid strain. Dominant mutants will either senesce due to the absence of telomerase or die even faster by the introduction of telomerase.

During the screening process, cells senesce because they are growing without telomerase. Importantly, mutants of interest cannot be rescued from senescence by activating telomerase since, by definition, they cannot tolerate this activity. There should be no more than 40 doublings before the colonies of interest are identified and rescued by mating to a wild type. This is based on an estimate of about 20 doublings to form a colony from a single cell and about 10 doublings to recreate a colony after replica plating. Considering that the screen is performed in a strain with very long telomeres viability is not decreased for at least 30-50 doublings. Over 9020 colonies were screened and as a result tol genes were discovered.

General information and databases containing yeast genome are available publicly and can be found for example on public websites such as for example: http://bioinformatics.weizmann.ac.il; http://ourworld.compuserve.com/homepages/C_Velten/yeast.htm; http:flwww.ncbi.nlm.nih.gov/Yeast; http://genome-www. stanford. edu/Saccharomyces/; genome-ftp.stanford.edu (directory/yeast/genome_seq); http://vectordb.atcg.com/vectordb/; http://www.mpimg-berlin-dahlem.mpg.de/˜andy/GN/S. cerevisiae/; or http://www.mips. biochem.mpg.de/proj/yeast, the content of which and links therein are incorporated herein by way of reference. Other databases exist as well and these databases and links therein to other websites are equally suitable for the purposes of this invention. The examples include but are not limited to Yeast GenBank (A collection of all GenBank sequences that are derived from Saccharomyces cerevisiae); Yeast Swiss-Prot (The collection of Swiss-Prot protein sequences that are derived from Saccharomyces cerevisiae); and YPD (The Yeast Protein Database maintained by Proteome, Inc.), periodic updates thereof the content of which is incorporated herein by way of reference. Methods of manipulating yeast are well established and are well known to those skilled in the art and can be found in publicly available web sites such as for example www.goshen.edu/bio/yeast, www.fhcrc.org/˜gottschling, and www.sacs.ucsf. edu/home/HerskowitzLab/protocols/protocol, among many others.

The invention also features a method of identifying mutant organisms having conditional-sensitive lethal mutations, and subsequently gene products thereof. The disclosed methods are useful for high-throughput screening of genomic or mutant libraries to rapidly identify genes, and corresponding gene products, which are essential for survival. A lethal mutation results in a gene or a protein which is not functional under restrictive conditions (i.e., in a tumor cell). A non-functional gene can have a defect in the promoter resulting in a reduced or abnormal gene expression. A non-functional protein can have a conformational defect causing improper protein folding or abnormal protein degradation. Improper protein folding can result in partial or total failure to fold, to recognize a native substrate, and/or to bind and release the substrate.

The invention pertains to novel compounds that are capable of inhibiting telomerase-dependent processes and are capable of regulating cell proliferation. Thus, a preferred use for the compounds of the invention is for inhibiting cell proliferation. In particular, the compounds of the invention can be used for treating a subject having an excessive or abnormal cell growth.

There are a wide variety of pathological cell proliferative conditions for which the compounds of the present invention can provide therapeutic benefits, with the general strategy being the inhibition of an anomalous cell proliferation. To illustrate, cell types which exhibit pathological or abnormal growth include various cancers and leukemias, psoriasis, bone diseases, fibroproliferative disorders such as involving connective tissues, atherosclerosis and other smooth muscle proliferative disorders, as well as chronic inflammation.

In addition to proliferative disorders, the treatment of differentiative disorders which result from, for example, de-differentiation of tissue which may (optionally) be accompanied by abortive reentry into mitosis. Such degenerative disorders include chronic neurodegenerative diseases of the nervous system, including Alzheimer's disease, Parkinson's disease, Huntington's chorea, amylotrophic lateral sclerosis and the like, as well as spinocerebellar degenerations. Other differentiative disorders include, for example, disorders associated with connective tissue, such as may occur due to de-differentiation of chondrocytes or osteocytes, as well as vascular disorders which involve de-differentiation of endothelial tissue and smooth muscle cells, gastric ulcers characterized by degenerative changes in glandular cells, and renal conditions marked by failure to differentiate, e.g. Wilm's tumors.

In addition to therapeutic applications (e.g., for both human and veterinary uses) it will be apparent that the subject compounds can be used as a cell culture additive for controlling proliferative and/or differentiation states of cells in vitro, for instance, by controlling the length of telomeres.

It will also be apparent that differential screening assays can be used to select for those compounds of the present invention with specificity for non-human yeast enzymes. Thus, compounds which act specifically on eukaryotic pathogens, e.g., are anti-fungal or anti-parasitic agents, can be selected from the subject inhibitors. To illustrate, inhibitors of the invention can be used in the treatment of candidiasis—an opportunistic infection that commonly occurs in debilitated and immunosuppressed patients. These same inhibitors could be used to treat these infections in patients with leukemias and lymphomas, in people who are receiving immunosuppressive therapy, and in patients with such predisposing factors as diabetes mellitus or AIDS, where fungal infections are a particular problem.

By way of illustration, the assays described in the art can be used to screen for agents which may ultimately be useful for inhibiting at least one fungus implicated in such mycosis as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocaidiosis, para-actinomycosis, penicilliosis, monoliasis, or sporotrichosis.

In addition to such therapeutic uses, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms.

In similar fashion, side by side comparison of inhibition of a mammalian gene and an insect gene, such as the Drosophilia, will permit selection amongst the subject derivatives of inhibitors which discriminate between the human/mammalian and insect enzymes. Accordingly, the present invention expressly contemplates the use and formulations of the subject in insecticides, such as for use in management of insects like the fruit fly.

In yet another embodiment, certain of the subject inhibitor compounds can be selected on the basis of inhibitory specificity for plant gene relative to the mammalian counterpart. For example, a plant gene can be disposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam.

The phrase “therapeutically-effective amount” as used herein means that amount of a compound, material, or composition comprising a compound of the present invention which is effective for producing some desired therapeutic effect by inhibiting an intracellular signalling pathway in at least a sub-population of cells in an animal and thereby blocking the biological consequences of that pathway in the treated cells, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present inhibitors may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, eye drops, powders, implants and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These peptides and compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compounds employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition).

The invention now being generally described, it will be more readily understood by reference to the following examples which are included merely for purposes of illustration of certain aspects and embodiments of the present invention, and are not intended to limit the invention.

### EXAMPLE 1

### Identification of Yeast Tol Genes by Telomerase-Dead Screen

The mutants, which are inviable in the presence of telomerase overexpression in yeast or vice versa, are screened as follows. The yeast strain used contains the EST1, EST2 and TEL1 genes under the inducible promoter from the GAL1 gene on circular centromeric plasmids. The strain is mutagenized to yield a 10-30% survival rate with either EMS or UV. Then the mutagenized cell titer is determined by plating 500 colony forming units of mutagenized cells per 120 mm Petri dish, on an agar-based media containing necessary salts, vitamins and dextrose. In two to three days, when the colonies have grown, the Petri dishes containing mutagenized colonies are replica-plated on three similar plates, having dextrose, galactose or glycerol as a carbon source. The colonies that did not grow on galactose-containing media, but grew on dextrose and glycerol-containing media are selected. Colonies formed by respiration-deficient cells will not grow on glycerol and thus are omitted from further analysis. The isolates are re-screened for the absence of growth on galactose-containing media.

The successful isolates are then allowed to lose the TLC1-containing plasmid. This should allow the mutant cells to grow on galactose-containing media. Then successful isolates are crossed with the wild type strain of the opposite mating type. If the hybrid strain grows on galactose, it is concluded that the synthetic lethal mutation in the isolate is recessive. If the hybrid strain cannot grow on galactose, the mutation is dominant and it is omitted from the successive analysis. The hybrids that contain recessive synthetic lethal mutations are crossed with the wild type strain, sporulated and dissected and the spore tetrads are checked for the appropriate single mutation pattern of segregation of the synthetic lethal phenotype. The mutants that show such pattern of segregation are cloned by complementation. Variations of this and other screening yeast methods are well known (see e.g., U.S. Pat. Nos. 5,912,154; 5,908,752; 5,876,951; 5,869,287; 5,866,338; 5,780.9,184; 5,674,996; 5,578,477; 5,527,896; 5,352,581; 5,175,091; and 5,139,936) and one skilled in the art readily knows which one to select for a specific purpose.

### EXAMPLE 2

### Identification of Mammalian and Human Homologue of Tol Genes

The strategy for identification of homologs and analogs is well known in the art and details can be for example found in PCT publication WO 99/27113, WO 99/01560, WO 98/45450 as incorporated herein by way of reference. In essence the strategy involves screening libraries of human or mammalian genome with a probe complementary to tol(s) cDNA. Methods of cloning, isolating, sequencing genes are well known in the art. As a result, among mammalian homologues of tol genes, the following genes are found, including but not limited to, CHL1, human helicase, ercc2 gene, mouse DNA helicase, human type II keratin subunit protein.

### EXAMPLE 3

### Method of Screening Cancer Using Tol Genes

The instant invention allows one skilled in the art using clinically obtainable body fluids, such as blood, plasma, lymph, pleural fluid, spinal fluid, saliva, sputum, urine, and semen, or tissue samples for example, to both detect the presence of cancer as well as assess the stage of the disease and the prognosis of a patient. The assay involving to genes or their products can be used as a diagnostic marker for malignancy as well as a means of monitoring the progress and effectiveness of cancer therapies. The approach of using clinically relevant genes is well known in the art and can be found for example in the following PCT publications as incorporated by reference, e.g., WO 99/41406; WO 99/40221; WO 99/35261; WO 98/59040; WO 99/33998; WO 97/35871; WO 97/28281; WO 98/37241; WO 98/37181; WO 98/28442; WO 98/21343; WO 98/14593; WO 98/14592; WO 98/11207; WO 98/08938; WO 98/07838; WO 98/02581; WO 98/01543; WO 98/01542; WO 98/00563; WO 97/41262; WO 97/20069; WO 97/18322; WO 97/15687; and WO 97/11198.

Frozen biopsies from a clinical lab are obtained and screened for human homologs of telomerase dysregulation by an RT-PCR method, which detects tol specific mRNA expression in samples. The choice of primers is decided based on available nucleic acid sequence data of tol genes. The following results are obtained: of eight normal breast tissue samples none had signal which would indicate positive signal (0/8); of four primary carcinoma one tested positive (1/4); of 17 ductal adenocarcinoma sixteen are positive (16/17) while in adjacent “normal tissue” only 2 are positive. Among samples of benign prostate hyperplasia those of advanced stage are tested positive.

### EXAMPLE 4

### Methods of Extending Lifespan

The present invention also relates to methods and reagents for extending the life-span, e.g., the number of mitotic divisions, of a normal cell. In general, the subject method relies on the instant discovery of tol genes in relation to telomerase catalytic subunit EST2, or a TLC1 or bioactive fragments thereof. The subject method is useful both in vivo, ex vivo and in situ. In essence it is discovered that while mutated tol genes are capable of causing senescence- or death of cells with high telomerase activity, the enhancement of normal non-mutated counterparts results in extending the lifespan of normal host cells. Transgenic animals are designed as disclosed hereinafter which are contemplated as a model to provide an insight into this discovery.

It is likely that overexpression of “normal” tol generates life-extending opportunity. When homozygous, tol1 produces an increase of mean and maximum life span of about 20% in Caenorhabditis elegans over that of the wild-type strain, N2.

The benefit of this discovery is significant, for the background see for example, PCT publication as incorporated herein by way of reference WO 99/35243.

### EXAMPLE 5

### Methods of Screening and Testing Agonists and Antagonists Inhibitors of Tot Genes/Products

The present invention identifies compounds that interact with instant genes and or products encoded by tol genes alone or in combination with telomerase genes and/or telomerase itself. As tol mutants when combined with overexpressed telomerase cause senescence and lethality, drugs that inhibit or suppress tol activity have the potential to kill tumor cells. In contrast, drugs that inhibit mutated tol activity and/or enhance wildtype tol activity are likely to be useful for increasing the number of cell replications and ultimately enhancing the cell lifespan. A model for screening such compounds based on yeast is established and in combination with known art techniques in providing extensive libraries of candidate compounds it is possible to identify compounds that exhibit activity in such a model. These compounds are shown to be effective and are contemplated to be useful in developing cancer treatments or prolonging lifespan. Drugs useful in treating cell cycle or DNA replication disorders identified in the present invention can now be screened using established yeast models as described herein above. Several classes of drugs can be screened such as chemical organic and inorganic compounds, peptides or peptide mimetics, antisense molecules, antibodies, etc. Methods of generating and performing high-throughput screening of chemical libraries are well established and are well known to those skilled in the art. Using the insights gained in the cell cycle regulation a drug discovery program is established to screen selected candidates in yeast-based assays and evaluate their therapeutic potential by further testing mammalian cells or cell lines. These pharmaceutical agents would ideally kill cells overexpressing telomerase in a predictable fashion, thereby enabling the determination of their future promise in cancer or other cell cycle related clinical conditions. Suitable compounds that can be tested include those disclosed in detail in the following PCT publications: WO 99/41262; WO 99/41261; WO 99/40087; WO 99/38964; WO 99/33861; WO 99/08679; WO 99/03507; WO 98/50397; WO 98/47911; WO 98/40080; WO 98/40066; WO 98/40065; WO 98/39966; WO 98/39965; WO 98/33503; WO 98/29114; WO 98/25885; WO 98/25884; WO 98/23759; WO 98/11204; WO 97/38013; WO 97/37691; and WO 97/02279.

Accordingly, a variety of libraries on the order of 1000 to 100,000 or more diversomers of the subject compounds can be synthesized, and, by use of a high throughput assay for detecting inhibitors, such as described in PCT publication WO 94/09135, rapidly screened for biological activity. For a review of methods of combinatorial synthesis, and methods of library screening and deconvolution, see, e.g., E. M. Gordon et al. (1994) J. Med. Chem. 37:1385-1401, and references cited therein. Furthermore, equally suitable antagonists such as tyrphostin, pyrozolopyrimidine and their derivatives and salts are contemplated as useful pharmaceutical compounds. The inhibitory activity of other drugs such H-89, K252a, H-7, N-(9-acridinyl)maleimide, staurosporine, herbimycin A, isoflavones like genistein, daidzein, quercetin (as disclosed in U.S. Pat. Nos. 5,919,813 and 5,872,223), quinolymethylen-oxindole (as disclosed in U.S. Pat. No. 5,905,149), angelmicin, 2-iminochromene derivatives (as disclosed in U.S. Pat. No. 5,648,378), 5-aminopyrazoles (as disclosed in U.S. Pat. No. 5,922,741) sesquiterpene lactone (as disclosed in U.S. Pat. No. 5,905,089), various benzylidene-Z-indoline compounds (as disclosed in U.S. Pat. No. 5,880,141), urea- and thiourea-type compounds (as disclosed in U.S. Pat. No. 5,773,459), benzopyran compounds (as disclosed in U.S. Pat. No. 5,763,470), polyhydric phenol compounds (as disclosed in U.S. Pat. No. 5,780,008), resorcyclic acid lactones (as disclosed in U.S. Pat. No. 5,674,892), 4-aminopyrrolo[2,3-d]pyrimidines (as disclosed in U.S. Pat. No. 5,639,757), and miscellaneous other phosphotyrosine phosphatase inhibitors (as disclosed in U.S. Pat. No. 5,877,210) is also tested in the same yeast assay system. It is thus clear that screening assay using instant mutants is extremely useful assay in identifying antagonist compounds targeting these particular targets.

The utility of this approach is further confirmed by utilizing a telomerase overexpressing fibroblast assay. While promoters and other conditions are different the principle is within the scope and spirit of instant invention. In this assay the compounds identified above are used to inhibit the proliferation of fibroblasts or specifically eliminate them. In addition to proliferation inhibition and killing effect, the expression of mutant gene is also monitored by standard art accepted methods aimed at testing useful drugs.

A library of peptides to be tested are synthesized according to the procedure disclosed in U.S. Pat. No. 5,532,167 to Cantley, which incorporated herein by way of reference. In addition to peptides as antagonists of instant mutants various other compounds are identified based on the assay disclosed above. These include but are not limited to antisense molecule, which is complimentary to the 5′ region of gene and blocks transcription via triplex formation. One skilled in the art may select appropriate oligonucleotide according to established procedures. For example, a series of methoxyethylamine 3′ end-cap oligodeoxynucleotides are prepared on a Biosearch 8750 DNA synthesizer, using standard H-phosphonate chemistry on controlled pore glass. The 15 or 18-base oligodeoxynucleotides are purified via DMT-on purification on a semi-prep Dynamax C-4 300A column. A secondary DMT-off purification is then performed on the same column. The oligomers are then desalted over a Pharmacia NAP-25 column, converted to the sodium form via Biorad AC 50W-X8 (Na⁺) 200400 mesh polyprep column, and then passed over another NAP-25 column. The antisense oligos and their controls, which contained the same bases but in scrambled sequence, are prepared in a similar manner. Lyophilized oligomers used in following experiments are dissolved in PBS (1 mM stock) and sterile filtered with Millipore 0.2 micrometer disks. The sequence used for antisense inhibitory studies on gene of interest is a 27 base region of the corresponding mRNA spanning the AUG translation initiation codon. While the present invention is not limited to such sequences, antisense oligonucleotides directed against the initiation codon region of the mRNA are one type of antisense molecule believed to effectively inhibit translation of the resulting gene product. Other effective antisense molecules can be specifically targeted against the opposite end of the mRNA.

To selectively interfere with the “normal” expression of dominant tol genes and to enhance expression of recessive tol genes interacting with overexpressed telomerase genes or products thereof, 5 mice are injected once with 5 μg/g weight of antisense, phosphorothioated oligodeoxynucleotide prepared as above and which is complementary to the initiator AUG domain in mRNA or with PBS for controls. Three weeks following the injection, liver biopsies are prepared from all of these mice. Each biopsy is frozen and then sliced into thin slices and hybridized with isotope labeled nucleic probes. Following 3 days of exposure to emulsion autoradiography, slides are developed to create silver grains over cells containing mRNAs of interest. Labeling and number of positive cells is decreased in liver specimens of mice treated with antisense phosphorothioated oligodeoxynucleotide demonstrating that antisense interferred with expression. In contrast, in control mice, mRNA levels per cell increased by about 2-fold. The decrease of expression is also confirmed by Western Blot studies using antibodies disclosed in Example 7.

The methods of selecting, making, administering, and testing appropriate doses of an antisense molecule along with suitable modifications, adjuvants and molecules are well known in the art and can be found for example in U.S. Pat. Nos. 5,734,039, 5,583,032, 5,756,476, 5,856,103, and 5,677,289 which are incorporated herein by way of reference. In addition to classical antisense molecule targeting AUG sequence one skilled in the art will know to use other suitable approaches such as a non-coding sense sequence, ribosomal frameshifting, and a ribozyme sequence. The details for such approaches can be found for example, in U.S. Pat. Nos. 5,843,723, 5,759,829, 5,707,866, and 5,712,384 as incorporated herein by way of reference. Without limiting to above anti-sense approaches it is clear that other means are equally suitable such as compositions and methods for the treatment of transformed malignant cells by antisense nucleic acid molecules that may cause the death of said cells such as disclosed in U.S. Pat. No. 5,935,937, which is incorporated herein by way of reference.

Drugs that are screened out as positive in yeast based assays and/or mammalian cell assays are then tested for treating diseases caused by excessive cell growth in animals. For this purpose Fisher 344 rats receive an inoculation of syngeneic 9 L gliosarcoma cells (4×104) into the deep white matter of the right cerebral hemisphere. The animals are subjected to two weeks of continuous treatment with test compound using osmotic minipumps transplanted subcutaneously. In control rats the minipumps are filled with saline. Statistical analysis of data employed the Fisher's Exact Test. Treatment of glioblastoma cells with the drug targeting telomerase overexpressing cancer cells results in time- and dose-dependent growth arrest and cell death accompanied by similarly diminished DNA synthesis. After 4-6 days of treatment, the extent of inhibition of tumor growth is assessed by comparison with control animals.

It is thus apparent that this approach is valid in treating a wide variety of diseases. Most of such diseases are malignant diseases, i.e., cancers of any of a wide variety of types, including without limitation, solid tumors and leukemias such as apudoma, choristoma, branchioma, malignant carcinoid syndrome, carcinoid heart disease, carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell), histiocytic disorders, leukemia (e.g., B-cell, mixed-cell, null-cell, T-cell, T-cell chronic, HTLV-II-associated, lymphocytic acute, lymphocytic chronic, mast-cell, and myeloid), histiocytosis malignant, Hodgkin's disease, immunoproliferative small, non-Hodgkin's lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcomas, neoplasms (e.g., bone, breast, digestive system, colorectal, liver, pancreatic, pituitary, testicular, orbital, head and neck, central nervous system, acoustic, pelvic, respiratory tract, and urogenital), neurofibromatosis, and cervical dysplasia.

Some diseases occur due to excessive but benign cell proliferation (i.e. non-malignant). Examples of such diseases are fibrosis, benign prostate hyperplasia, atherosclerosis, restenosis, glomulerosclerosis, cheloid, psoriasis, lentigo, keratosis, achrochordon, molluscum contagiosum, venereal warts, sebaceous hyperplasia, condylomata acuminatum, angioma, venous lakes, chondrodermatitis, granuloma pyogenicum, hidradenitis suppurativa, keloids, keratoacanthoma, leukoplakia, steatocystoma multiplex, trichiasis, superficial epithelial nevus, polyp, junctional nevus, pyogenic granuloma, prurigo nodularis, dermatofibroma, adenoma sebaceum, and other diseases of the skin and non-malignant neoplastic diseases such as for example Kaposi's sarcoma, papilloma.

In a similar manner it thus obvious that antagonists of tol activity will display utility in prolonging cell life and are useful for preventing senescence.

### EXAMPLE 6

### Transgenic Animals Having Mutated Tol Genes

Method of making transgenic nonhuman organisms, such as transgenic mice, with tol genes is provided. The strategy of making such animals is well known in the art and can be found for example in WO 97/35967 which is incorporated herein by way of reference. Methods of using these organisms, including methods of detecting compounds that affect the tumors or are useful for geriatric studies are contemplated and provided.

These methods are essentially same as disclosed herein. According to the present invention, transgenic animals of any non-human species, including but not limited to mice, rats, rabbits, guinea pigs, pigs, or non-human primates may be produced using any technique known in the art, including but not limited to microinjection, electroporation, cell gun, cell fusion, or functional equivalents (see U.S. Pat. No. 5,550,316). In preferred embodiments of the invention, transgenic animals are generated according to the method disclosed hereinafter. Briefly, this method entails the following. Transgenic offspring is prepared by microinjecting a recombinant nucleic acid construct into fertilized eggs. For example, and not by way of limitation, fertilized mouse eggs may be collected from recently mated females with vaginal plugs, and then microinjected with construct DNA. Construct DNA, at a concentration of about 0.01-3 g/ml, is microinjected into the male pronucleus of fertilized eggs, in an amount such that the volume of the pronucleus approximately doubles. The injected eggs is then be transferred to female mice which had been mated the night before with vasectomized males. See also U.S. Pat. No. 4,873,191 by Wagner and Hoppe. DNA clones for microinjection are cleaved with appropriate restriction enzymes, such as Sal1, Not1, etc., and the DNA fragments electrophoresed on 1% agarose gels in TBE buffer (U.S. Pat. No. 5,811,633). The DNA bands are visualized by staining with ethidium bromide, excised, and placed in dialysis bags containing 0.3M sodium acetate at pH 7.0. The DNA is then electroeluted into the dialysis bags, extracted with phenol-chloroform (1:1), and precipitated by two volumes of ethanol. The DNA is redissolved in 1 ml of low salt buffer (0.2M NaCl, 20 mM Tris, pH 7.4, and 1 mM EDTA) and purified on an Elutip-D column. The column is first primed with 3 ml of high salt buffer (1M NaCl, 20 mM Tris, pH 7.4, and 1 mM EDTA) followed by washing with 5 ml of low salt buffer. The DNA solutions are passed through the column for three times to bind DNA to the column matrix. After one wash with 3 ml of low salt buffer, the DNA is eluted with 0.4 ml of high salt buffer and precipitated by two volumes of ethanol. DNA concentrations are measured by absorption at 260 nm in a UV spectrophotometer. For microinjection, DNA concentrations are adjusted to about 3 g/ml in 5 mM Tris, pH 7.4 and 0.1 mM EDTA. Other methods for purification of DNA for microinjection are also known. The purified inserts form plasmids are then microinjected into the pronuclei of fertilized (C57BU6×CBA)F2 mouse embryos and surviving embryos are transferred into pseudopregnant females according to standard procedures such as disclosed in U.S. Pat. Nos. 5,877,397, 5,907,078, 5,849,993, 5,602,309, 5,387,742, which are incorporated herein by way of reference. Construct is operably linked to a suitable promoter, e.g., RSV long terminal repeat (LTR), glial fibrillary acidic protein (GFAP), or human beta-globin promoter (GF). Mice that developed from injected embryos are analyzed for the presence of transgene sequences by Southern blot analysis of mutant DNA. Transgene copy number is estimated by band intensity relative to control standards containing known quantities of cloned DNA. At 3 to 8 weeks of age, cells are isolated from these animals and assayed for the presence of transgene encoded tol products. All of the control non-transgenic mice tested negative for expression of mutated tol. Southern blot analysis indicates that many of these mice contain one or more copies of the transgene per somatic and/or germ cell. These mice are useful as a model for studying tol mutants in vivo for testing, for example, tol agonists or antagonists.

### EXAMPLE 7

### Immunization Approach Based on Instant Invention

Examples listed above show that under certain circumstances telomerase overexpressing cells display lethality by providing tol mutants. A similar principle may be operative in cancer growth wherein tol genes are not operative and cancer expressing high telomerase levels mat replicate indefinitely. It thus likely that normal cells will differ from cancer cells due to different antigenicity of tol. If immune system of a host is primed to recognize and distinguish such antigens these malignant cells can be eliminated.

A group of three Balb/c female mice (Charles River Breeding Laboratories, Wilmington, Mass.) are injected with 5 g/dose of substantially purified products of telomerase enzyme and other mutant tol proteins in 100 l Detox adjuvant (RIBI ImmunoChem Res Inc, Hamilton, Mo.) by intraperitoneal injection on days 0, 3, 7, 10, and 14. On day 17 the animals are sacrificed, their spleens are removed and the lymphocytes fused with the mouse myeloma line 653 using 50% polyethylene glycol 4000 by an established procedure (see U.S. Pat. Nos. 5,939,269, and 5,658,791 as incorporated herein by way of reference). The fused cells are plated into 96-well microtiter plates at a density of 2×10⁵ cells/well followed by HAT selection on day 1 post-fusion. Immobilized hybridoma culture supernatants are then reacted with biotinylated mutant pp60 C-terminal peptide. The wells positive for antibodies are expanded for further study. These cultures remain stable when expanded and cell lines are cryopreserved. The parental cultures are isotyped and then assayed for their ability to capture and to specifically recognize mutant protein complexes. These complexes are then tested in tumor models as immunogens that raise specific immune response.

Alternatively, polyclonal rabbit antisera is raised against purified mutant protein peptides. Polyclonal antibodies against the C-terminal peptide are obtained by coupling such peptides to Keyhole Limpet Heamocyanin with 0.05% gluteraldehyde, emulsified in Freunds' complete adjuvant and injected intradermally at several sites. The animals are boosted four and seven weeks later with coupled peptide emulsified in Freunds' incomplete adjuvant and bled ten days after the last injection.

Antibodies prepared according to above procedures are then used for identifying and/or diagnosing tumor cells that overexpress telomerase and tol complexes and/or for therapeutic approaches according to standard procedures known in the art, e.g., U.S. Pat. Nos. 5,601,989, 5,563,247, 5,610,276, and 5,405,941, as incorporated herein by way of reference.

### EXAMPLE 8

### Method of Treating Yeast Infection

As described above, these compounds targeting specifically cells with abundant telomerase can be used effectively by women to control yeast infection without upsetting the microfloral balance of the vagina. The compounds may similarly be used to control Candida microbes around wounds. Other yeast organisms are equally suitable as targets of treatment including but not limited to Candida albicans, Candida stellatoidea, Candida tropicalis, Candida parapsilosis, Candida krusei, Candida pseudotropicalis, Candida quillermondii, Candida rugosa, Aspergillus fumigatus, Aspergillus flavus, Aspergillus niger, Aspergillus nidulans, Aspergillus terreus, Rhizopus arrhizus, Rhizopus oryzae, Absidia corymbifera, Absidia ramosa, and Mucor pusillus or combination thereof.

In this study 5 affected patients apply topically drug-containing vaginal suppositories once a day. As a result no yeast infection is observed in any of the patient after period of three days. Also, no untoward reactions of any kind are evident in any of these patients. It should be understood in this regard, that topical use in accordance with the present invention is not limited to the areas to be treated. The pharmaceutical composition of the present invention is equally suitable for treating or preventing yeast infections when delivered by other means, e.g., orally, intravenously, etc.

All of the above-cited internet sources, patents, publications, and references within are hereby expressly incorporated by way of reference.

The invention being thus described, it will be obvious that the same may be varied in many ways. Such variations are not to be regarded as a departure from the spirit and scope of the invention, and all such modifications are intended to be included within the scope of the following claims.

1. A method of identifying a secondary target site comprising:
(a) providing a plurality of cells having a genome, which includes at least one primary gene encoding telomerase activity and a promoter that can direct the over expression of said primary gene;
(b) effecting one or more mutations in the genome of said cells, at one or more secondary sites;
(c) selecting those cells having at least one mutation that proves lethal to said cells when said primary gene is over expressed;
(d) determining a site in the genome of said cells in which said at least one lethal mutation is located, to provide a secondary target site.

2. The method of claim 1 in which said secondary target site includes a tot gene, a homolog thereof, or an analog thereof, including mammalian homologs or analogs thereof.

3. The method of claim 2 in which said tot gene is selected from the group consisting of tol1, tol2, tol3, a homolog thereof, or an analog thereof, including mammalian homologs or analogs thereof.

4. The method of claim 1 in which said secondary target site or any gene product thereof is involved in either the modulation of the expression of said primary gene or a process affecting the viability of the cell in which said primary gene is over expressed.

5. The method of claim 1 in which said at least one primary gene is selected from the group consisting of EST1, EST2, EST3, TLC1, a homolog thereof, an analog thereof, including mammalian homologs or analogs thereof, or combinations thereof.

6. The method of claim 2 in which a homolog or an analog of said tol gene is selected from the group consisting of CHL1, a gene encoding human helicase, ercc2, a gene encoding mouse DNA helicase, or a gene encoding human type II keratin subunit protein.

7. The method of claim 5 in which said TLC1 gene is selected from the group consisting of embZ35904, gbUl4595, embZ35905, dbjD28120, gbL24113, embX76992, gbACOO5476.3, gbU53340, including mammalian homologs or analogs thereof, or combinations thereof.

8. The method of claim 5 in which said EST1, EST2, and EST3 genes are selected from those genes encoding human kininogen HMW heavy chain, prepro alpha-2thio]proteinaise, calmodulin-stimulated protein, kininogen, immunoglobulin kappa chain, nitric-oxide synthase, immunoglobulin heavy chain variable, T-cell receptor deltachain V, Ig gamma-chain, Ig H-chain V-D-JH4-region, perlecan, insulin-like growth factor II, interferon-alpha, rat coding sequence of p15 and p12, interferon-alpha I precursor, AAD 1 0, or combinations thereof.

9. The method of claim 1 which further comprises using said secondary target site, or lethal mutations thereof, to screen for a drug or drug candidate.

10. The method of claim 9 in which said drug or drug candidate inhibits the growth or replication of a human tumor or causes the demise of said human tumor.

11. The method of claim 9 in which said drug or drug candidate interacts with, binds to, or inhibits the expression or activity of a gene product associated with said secondary target site.

12. The method of claim 9 in which said drug or drug candidate comprises a polypeptide, an oligonucleotide, a polysaccharide, or a small molecule.

13. A secondary target site comprising a site in the genome of a cell, which genome includes at least one primary gene encoding telomerase activity and a promoter that can direct the over expression of said primary gene, said site able to accommodate at least one mutation that can prove lethal to said cell when said primary gene is over expressed.

14. The lethal mutations of the secondary target site of claim 13 and allelic variations thereof.

15. A method of inhibiting the growth or replication of a tumor cell or causing the demise of said cell, said cell exhibiting aberrant telomerase activity, comprising administering a drug or drug candidate that interacts with, binds to, or inhibits the expression or activity of a gene product associated with a secondary target site in the genome of said cell, which site can accommodate at least one mutation that can prove lethal to said cell.

16. The method of claim 15 in which a secondary target site includes a tol gene, a homolog thereof or an analog thereof, including mammalian homologs or analogs thereof.

17. The method of claim 15 in which said tol gene is selected from the group consisting of tol1, tol2, toB, a homolog thereof, or an analog thereof, including mammalian homologs or analogs thereof.

18. The method of claim 15 in which said aberrant telomerase activity comprises overexpression of telomerase.

19. A pharmaceutical composition comprising an effective amount of a drug or drug candidate and a pharmaceutically acceptable carrier or diluent, said drug or drug candidate capable of interacting with, binding to, or inhibiting the expression or activity of a gene product associated with a secondary target site in the genome of a cell exhibiting aberrant telomerase activity, which site can accommodate at least one mutation that can prove lethal to said cell.

20. A pharmaceutical composition of claim 19 in which said aberrant telomerase activity comprises overexpression of telomerase.

21. A recombinant eukaryotic cell comprising at least one secondary target site, a homolog thereof, or an analog thereof and at least one primary gene, a homolog thereof, or an analog thereof, wherein said at least one primary gene encodes telomerase and said at least one secondary target site contains a mutation such that the up regulation, down regulation, elimination, or disruption of said at least one primary gene gives rise to senescence or synthetic lethality.

22. A method of screening for drugs, comprising:
(a) providing one or more eukaryotic cells capable of telomerase overexpression, which one or more cells exhibit senescence or synthetic lethality under conditions of telomerase overexpression when a wild-type tol gene, its homolog, or its gene product of said one or more cells is mutated;
(b) contacting said one or more cells with one or more drug candidates under conditions that provide telomerase overexpression and, optionally, inhibition or mutation of said wild-type tol gene, its homolog, or its gene product; and
(c) selecting those drug candidates that give rise to senescence or synthetic lethality under conditions that provide telomerase overexpression or those drug candidates that inhibit, suppress, reverse, or prevent senescence or synthetic lethality under conditions that provide telomerase overexpression and inhibition or mutation of said wild-type tol gene, its homolog, or its gene product.

23. A secondary target site identified by the method of claim 1.

24. A method of inhibiting the growth or replication of a tumor cell or causing the demise of said cell, said cell exhibiting aberrant telomerase activity, comprising administering a drug or drug candidate that interacts with, binds to, or inhibits (or enhances) the expression or activity of a gene product associated with the secondary target site of claim 21.

25. A pharmaceutical composition comprising an effective amount of a drug or drug candidate and a pharmaceutically acceptable carrier or diluent, said drug or drug candidate capable of interacting with, binding to, or inhibiting (or enhancing) the expression or activity of a gene product associated with the secondary target site of claim 21.

26. The pharmaceutical composition of claim 25 in which said secondary target site is found in the genome of a cell exhibiting aberrant telomerase activity.

27. The pharmaceutical composition of claim 25 in which said aberrant telomerase activity comprises overexpression of telomerase.

28. A method of inhibiting the growth or replication of a tumor cell or causing the demise of said cell, said cell exhibiting aberrant telomerase activity, comprising administering a drug or drug candidate that interacts with, binds to, or inhibits (or enhances) the expression or activity of a gene product associated with a secondary target site.

29. A pharmaceutical composition comprising an effective amount of a drug or drug candidate and a pharmaceutically acceptable carrier or diluent, said drug or drug candidate capable of interacting with, binding to, or inhibiting the expression or activity of a gene product associated with a secondary target site in the genome of a cell exhibiting aberrant telomerase activity.

30. The pharmaceutical composition of claim 29 in which said aberrant telomerase activity comprises overexpression of telomerase.
